(19) United States
(12) Patent Application Publication
(10) Pub. No.: US 2026/0363550 A1
(43) Pub. Date: Nov. 19, 2026

(54) SMART CABLE INTERFACE SYSTEMS FOR USE IN MONITORING AND VISUALIZING MEDICAL SENSOR DATA

(71) Applicant: KONINKLIJKE PHILIPS N.V., EINDHOVEN (NL)

(72) Inventor: Hans Willem van Kesteren, Eindhoven (NL)

(21) Appl. No.: 19/124,330

(22) PCT Filed: Oct. 18, 2023
(86) PCT No.: PCT/EP2023/079010

§ 371 Date: Apr. 25, 2025

Related U.S. Application Data
(60) Provisional application No. 63/418,987, filed on Oct. 25, 2022.

(51) Int. Cl. A61B 5/00; G16H 40/67

(52) U.S. Cl. CPC A61B 5/0002; A61B 5/742; G16H 40/67; A61B 2562/06; A61B 2562/222; A61B 2562/227

(57) ABSTRACT

The present disclosure relates to the patient monitoring arts and provides systems and methods of monitoring one or more patients that are transitioning from one physical environment to another physical environment. The patient monitoring systems of the present disclosure include an interface device comprising one or more universal ports and a networking component, wherein the interface device is configured to receive sensor data from one or more physiological sensors attached to the patient via one or more smart cables, and wherein the networking component is configured to transmit the sensor data to a remote data storage unit. In accordance with the systems and methods of the present disclosure, a patient may be seamlessly transitioned from a typically high-acuity environment to a typically low-acuity environment.

### FIELD OF THE DISCLOSURE

The present disclosure relates generally to smart cable interface systems and methods of using such smart cable interface systems, and more specifically to smart cable interface systems and methods for use in monitoring and visualizing medical sensor data.

### BACKGROUND

Sophisticated patient monitors and systems for monitoring the health of a patient, such as patient monitors used for visualizing sensor data, are traditionally expensive. Further, in such systems, each sensor attached to a patient is connected to a control unit by a cable. The extensive wiring and cabling in such systems limits the patient's mobility and function, reduce patient comfort, and hinders access to the patient. Furthermore, at lower acuity care settings, continuous data visualization is frequently unnecessary.

### SUMMARY OF THE DISCLOSURE

According to an embodiment of the present disclosure, a system for monitoring sensor data associated with a patient is provided. The system comprises: one or more physiological sensors; one or more smart cables, each smart cable comprising a universal connector; and an interface device configured to receive sensor data from the one or more physiological sensors via the one or more smart cables, the interface device including one or more universal ports configured to receive a corresponding universal connector of the one or more smart cables.

In an aspect, each of the one or more the smart cables comprises a controller configured to enable communication of sensor data from a plurality of different sensor types.

In an aspect, the one or more physiological sensors include at least a first sensor having a first sensor type and a second sensor having a second sensor type, wherein the first sensor type is different from the second sensor type.

In an aspect, each of the one or more physiological sensors are connected to the universal port of the interface device via a universal connector of one of the one or more smart cable.

In an aspect, the one or more physiological sensors includes at least one of a pulse oximetry sensor, an ECG sensor, a heart rate sensor, a respiration rate sensor, a body temperature sensor, an actigraphy sensor, and a capnography sensor.

In an aspect, the system further comprises a remote data storage unit in wireless communication with the interface device, wherein the interface device further comprises a networking component configured to transmit the sensor data, and wherein the remote data storage unit is configured to receive the sensor data transmitted from the interface device and store the received sensor data in a memory.

In an aspect, the system further comprises a client device in communication with the remote data storage unit, the client device comprising: a networking component configured to receive patient information for a plurality of patients from the remote data storage unit; and a display configured to display the patient information received from the remote data storage unit; wherein the patient information includes sensor data for the plurality of patients stored in the memory of the remote data storage unit.

In an aspect, the interface device further comprises a global positioning system receiver configured to determine a location of the interface device, and wherein the networking component is further configured to transmit the location of the interface device to the remote data storage unit.

In an aspect, the one or more physiological sensors are configured to be attached to the patient and collect: (i) a first set of sensor data for the patient over a first period of time while connected to a first patient monitor in proximity to the patient; and (ii) a second set of sensor data for the patient over a second period of time while connected to the interface device.

According to another embodiment of the present disclosure, a method for monitoring a patient via one or more physiological sensors attached to the patient, while transitioning from a first environment to a second environment, is provided. The method comprises: in the first environment, collecting, via the one or more physiological sensors, a first set of sensor data for the patient over a first period of time, wherein the one or more physiological sensors are connected to a first patient monitor in proximity to the patient; and in the second environment, collecting, via the one or more physiological sensors, a second set of sensor data for the patient over a second period of time, wherein the one or more physiological sensors are connected to an interface device via one or more smart cables.

In an aspect, the method further comprises: transmitting, via the interface device, the second set of sensor data to a remote data storage unit; and storing, in a memory of the remote data storage unit, at least the second set of sensor data, wherein the memory of the remote data storage unit comprises patient information for a plurality of patients.

In an aspect, the method further comprises: receiving, at a client device, patient information from the remote data storage unit, wherein the patient information includes at least the second set of sensor data for the patient; and visualizing, on a display of the client device, the patient information.

In an aspect, the method further comprises: receiving, via a user input device of the client device, a request for the patient information of the patient, wherein the patient is selected from a plurality of patients for whom patient information is stored in the remote data storage unit; and transmitting, from the remote data storage unit, the patient information for the patient in response to receiving the request.

In an aspect, the method further comprises: adjusting a time stamp associated with sensor data of the second set of sensor data collected using at least one of the one or more physiological sensors, wherein the time stamp of the sensor data is adjusted based on a sensor-specific delay; and visualizing, on a display of the client device, the patient information, wherein the patient information includes the sensor data of the second set of sensor data having the adjusted time stamp.

In an aspect, the one or more physiological sensors includes at least a first sensor having a first sensor type and a second sensor having a second sensor type that is different from the first sensor type, and wherein the second set of sensor data comprises sensor data from at least the first and second sensors collected by the interface device via the one or more smart cables.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a diagram of a patient monitoring system illustrated according to aspects of the present disclosure.

FIG. 2 is a schematic block diagram of an interface device controller illustrated according to aspects of the present disclosure.

FIG. 3 is a flowchart of a method for monitoring a patient transitioning from a first environment to a second environment illustrated according to aspects of the present disclosure.

FIG. 4 is another diagram illustrating a patient monitoring system according to further aspects of the present disclosure.

### DETAILED DESCRIPTION OF EMBODIMENTS

The present disclosure is directed to patient monitoring systems and methods of monitoring a patient. More specifically, the present disclosure is directed to patient monitoring systems and methods employing an interface device that facilitates transition of the monitored patient between different environments while maintaining monitoring accuracy and continuity.

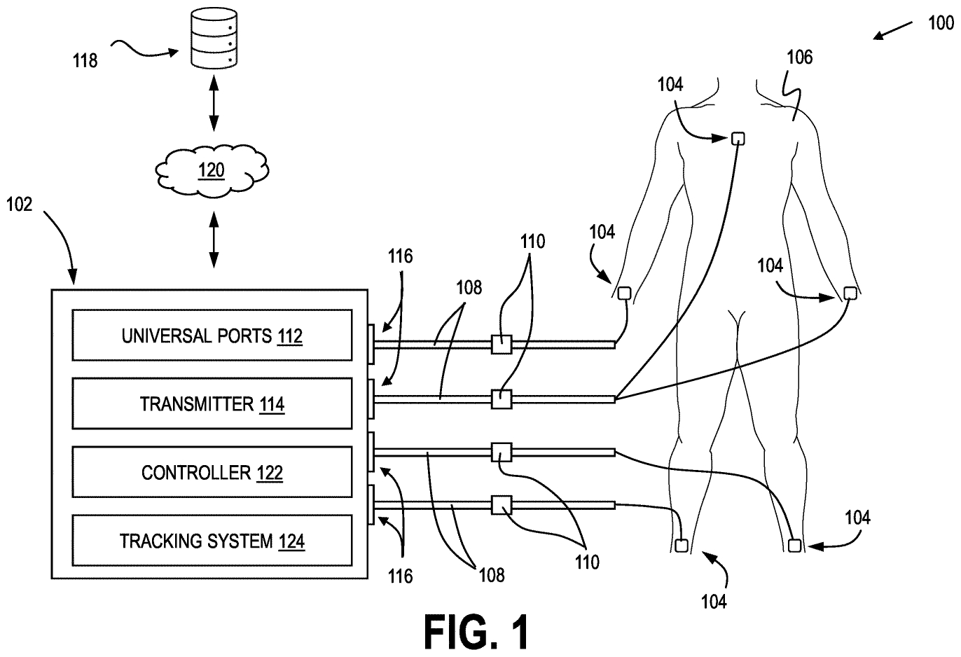

Turning to FIG. 1, a patient monitoring system 100 comprising an interface device 102 is illustrated according to aspects of the present disclosure. In embodiments, the interface device is configured to receive sensor data from one or more physiological sensors 104 attached to a patient 106. In embodiments, each physiological sensor 104 may be connected to the interface device 102 via a corresponding smart cable 108. In further embodiments, two or more physiological sensors 104 may be connected to the interface device 102 via a single smart cable 108. As used herein, the term “smart cable” refers to a medical cable that is connected to one or multiple different types of sensors, and may communicate signals measured using one or more different sensors. In embodiments, the smart cable 108 can include electronics 110 (e.g., a controller) configured to enable the communication of sensor data, through the cable 108, and to the interface device 102. In embodiments, the electronics 110 of each smart cable 108 may enable communication of sensor data from multiple different sensor types.

In embodiments, the interface device 102 comprises one or more of universal ports 112 and a networking component 114. The universal ports 112 can be configured to receive universal connectors 116 of the smart cables 108. Put another way, each of the smart cables 108 comprises a universal connector 116 configured to enable communication from one or more different sensor types to the interface device 102 via one or more universal ports 112.

The networking component 114 of the interface device 102 can be configured to transmit information from the interface device 102 to a remote data storage unit 118. For example, the networking component 114 can include a radio-frequency (RF) transmitter configured to generate RF waves according to one or more standardized protocols (e.g., IEEE 802.11, Bluetooth, etc.) in order to transmit patient information and/or sensor data. In particular embodiments, the networking component 114 may be configured to communicate patient information and/or sensor data over a particular type of network 120. In some embodiments, the network 120 may include one or more forms of wired and/or wireless communication.

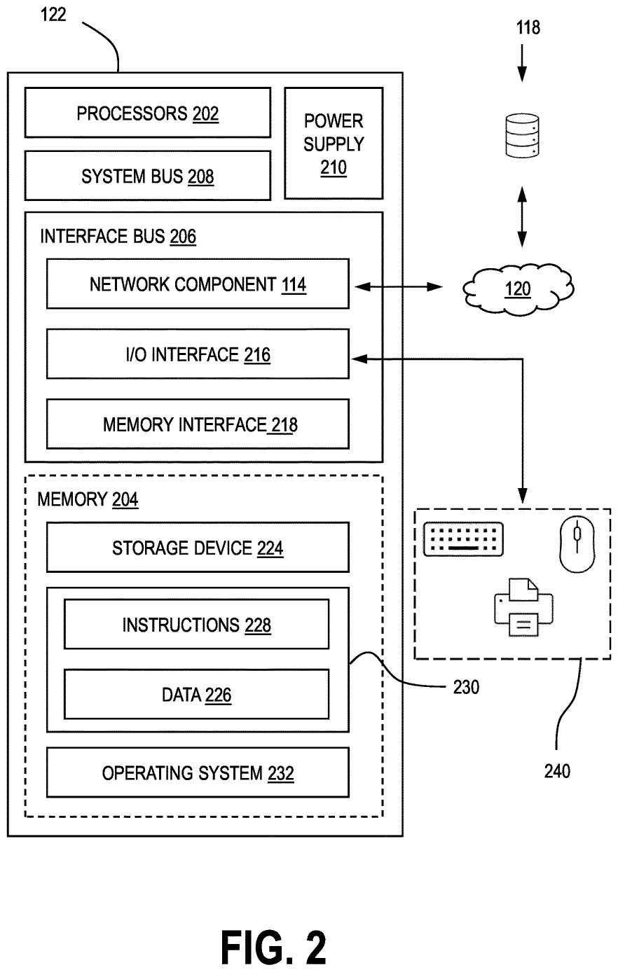

In particular embodiments, the networking component 114 may be a standalone component or may be part of a controller 122. Put another way, the interface device 102 may include a controller 122 comprising the networking component 114. For example, with reference to FIG. 2, a controller 122 that may form a part of the interface device 102 is illustrated according to aspects of the present disclosure. In embodiments, the controller 122 may include a networking component 114 and may be configured to communicate information to a remote data storage unit 118. In further embodiments, the controller 122 may be configured to operate the interface device 102 as described herein.

In the example of FIG. 2, the controller 122 can include one or more processors 202, machine-readable memory 204, and an interface bus 206, all of which may be interconnected and/or communicate through a system bus 208 containing conductive circuit pathways through which instructions (e.g., machine-readable signals) may travel to effectuate communication, tasks, storage, and the like. The controller 122 may be connected to a power source 210, which can include an internal power supply and/or an external power supply. In embodiments, the power source 210 powers the entire interface device 102.

The one or more processors 202 may include a high-speed data processor adequate to execute the program components described herein and/or various specialized processing units as may be known in the art. In some examples, the one or more processors 202 may be a single processor, multiple processors, or multiple processor cores on a single die.

In some examples, the interface bus 206 may include the networking component 114 as described herein, an input/output (“I/O”) interface 216 configured to connect and communicate with one or more peripheral devices 240, and/or a memory interface 218 configured to accept, communication, and/or connect to a number of machine-readable memory devices (e.g., memory 204).

The I/O interface 216 may operatively connect the controller 122 with one or more peripheral devices 240 via one or more wired and/or wireless connections. In some examples, the peripheral devices 240 may include, but are not limited to, a keyboard, a barcode or QR code scanner, a NFC card reader, a touch screen configured to receive alphanumeric input, a photo-optical keyboard, or other alphanumeric input components, a mouse, a touchpad, a trackball, a joystick, a motion sensor, or other pointing instrument, a physical button, a touch screen that provides location and/or force of touches or touch gestures, or other tactile input components, a microphone, and/or the like. In further examples, the peripheral devices 240 can include a client device, such as mobile phone, desktop computer, laptop, portable digital assistants (PDAs), smart phones, tablets, netbooks, laptops, multi-processor systems, microprocessor-based or programmable consumer electronics, game consoles, set-top boxes, or other communication device that a user may use to access a network. One or more peripheral devices 240 may, for instance, be used transmit and/or store patient identification information in the interface device 102 before subsequent acquisition of sensor data.

The networking component 114 may operatively connect the controller 122 to a communications network 120, which can include a direct interconnection, the Internet, a local area network (“LAN”), a metropolitan area network (“MAN”), a wide area network (“WAN”), a wired or Ethernet connection, a wireless connection, and similar types of communications networks, including combinations thereof. In some examples, controller 122 may communicate with one or more remote/cloud-based servers 118, cloud-based services, and/or remote client devices via the communications network 120 and the networking component 114.

The memory 204 can be variously embodied in one or more forms of machine-accessible and machine-readable memory. In some examples, the memory 204 includes a storage device 224 comprises one or more types of memory. For example, the storage device 224 can include, but is not limited to, a non-transitory storage medium, a magnetic disk storage, an optical disk storage, an array of storage devices, a solid-state memory device, and the like, including combinations thereof.

Generally, the memory 204 is configured to store data/information 226 and instructions 228 that, when executed by the one or more processors 202, causes the controller 122 to perform one or more tasks. The memory 204 and/or the storage device 224 may also be used to store patient identification, sensor data, and the like.

In particular examples, the memory 204 includes an interface device package 230 that comprises a collection of program components, database components, and/or data. Depending on the particular implementation, the interface device package 230 may include software components, hardware components, and/or some combination of both hardware and software components.

The interface device package 230 may include, but is not limited to, instructions 228 having one or more software packages configured to receive sensor data. These software packages may be incorporated into, loaded from, loaded onto, or otherwise operatively available to and from the controller 122.

In particular embodiments, the interface device package 230 can include, but is not limited to, instructions 228 configured to operate the interface device 102, receive sensor data from one or more physiological sensors 104, and/or transmit the received sensor data to a remote data storage unit 118. In embodiments, the interface device 102 may also include a location-based tracking system 124, and the controller 122 may be further configured to determine a location of the interface device 102. For example, the location-based tracking system 124 may be a global positioning system (GPS) module, and the interface device package 230 can include instructions 228 configured to operate the GPS module. These instructions 228 may be incorporated into, loaded from, loaded onto, or otherwise operatively available to and from the interface device 102.

The controller 122 may also include an operating system component 232, which may be stored in the memory 204. The operating system component 232 may be an executable program facilitating the operation of the controller 122. Typically, the operating system component 232 can facilitate access of the I/O interface, networking component, and memory interface, and can communicate with other components of the interface device 102.

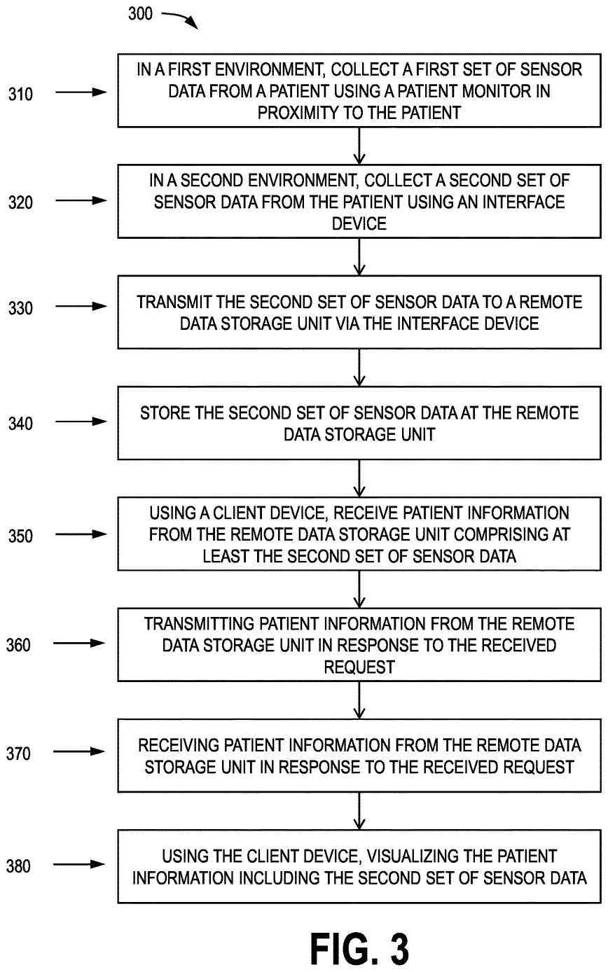

Also provided herein are methods 300 of monitoring a patient via one or more physiological sensors attached to the patient, while transitioning from a first environment to a second environment. With reference to FIG. 3, one such method 300 is illustrated according to aspects of the present disclosure. In the example of FIG. 3, the method 300 includes: in a step 310, collecting a first set of sensor data for the patient within the first environment over a first period of time via the one or more physiological sensors, wherein the one or more physiological sensors are connected to a first patient monitor in proximity to the patient; and in a step 320, collecting a second set of sensor data for the patient within the second environment over a second period of time via the one or more physiological sensors, wherein the one or more physiological sensors are connected to an interface device via a smart cable.

According to the step 310, the method 300 includes collecting sensor data for the patient over a first period of time (e.g., seconds, minutes, hours, days, weeks, etc.) via the one or more physiological sensors attached to the patient. The sensor data collected in the step 310 may be a first set of sensor data collective via the one or more physiological sensors. In some embodiments, the one or more physiological sensors may be attached to the patient while the patient is within the first environment, such as by a healthcare provider (e.g., physician, clinician, nurse, etc.). As such, the one or more physiological sensors may be expertly attached to the patient by an experienced operator. In further embodiments, the one or more physiological sensors may be connected to a patient monitor in proximity to the patient within the first environment. As such, the step 310 may further include visualizing, outputting, and/or otherwise analyzing the first set of sensor data locally to the patient within the first environment.

In embodiments, the first environment is a setting where the patient may receive experienced healthcare-related services. For example, and without limitation, the first environment may include a hospital, a doctor's office, or the like.

In embodiments, the one or more physiological sensors 104 can comprise multiple or a plurality of physiological sensors 104. In some embodiments, the one or more physiological sensors 104 include at least two different types of sensors. For example, the one or more physiological sensors 104 can include at least a first sensor having a first sensor type and a second sensor having a second sensor type that is different from the first sensor type. In some embodiments, the one or more physiological sensors 104 includes at least one of a pulse oximetry sensor, an electrocardiogram (ECG) sensor, a heart rate sensor, a respiration rate sensor, a body temperature sensor, an actigraphy sensor, and a capnography sensor. In further embodiments, the one or more physiological sensors 104 are high-acuity sensors that are positioned and/or attached to the patient 106 by an expert (e.g., nurse, physician, clinician, doctor, or other healthcare specialist), as discussed herein.

According to the step 320, the method 300 also includes collecting sensor data for the patient over a second period of time (e.g., seconds, minutes, hours, days, weeks, etc.) via the one or more physiological sensors attached to the patient. The sensor data collected in the step 320 may be a second set of sensor data collective via the one or more physiological sensors. The second set of sensor data may be collected from the patient via the one or more physiological sensors while the patient is in a second environment that is different than the first environment. In embodiments, the one or more physiological sensors 104 may be connected to an interface device 102 via one or multiple smart cables 108 while the patient is within the second environment.

As described herein, the one or more physiological sensors 10 can comprise multiple or a plurality of physiological sensors 104. Thus, in embodiments, the second set of sensor data may include sensor data from at least a first physiological sensor having a first sensor type and a second physiological sensor having a second sensor type that is different from the first sensor type. In some embodiments, the one or more physiological sensors 104 includes at least one of a pulse oximetry sensor, an ECG sensor, a heart rate sensor, a respiration rate sensor, a body temperature sensor, an actigraphy sensor, and a capnography sensor. In further embodiments, the one or more physiological sensors 104 are the same high-acuity sensors that are used to collect the first set of sensor data. Put another way, the one or more physiological sensors 104 used to collect the first set of sensor data from the patient within the first environment are the same physiological sensors 104 used to collect the second set of sensor data from the patient within the second environment.

In embodiments, the one or more physiological sensors 104 may be connected to an interface device 102 via one or multiple smart cables 108, as described herein, while collecting the second set of sensor data from the patient 106 within the second environment.

In embodiments, the second environment can be a location where the patient cannot be monitored via a conventional patient monitor. For example, while a first environment such as a hospital room or a doctor's office may have a patient monitor available, the patient monitor may not be able to travel or move around with the patient between locations. Instead, prior to the patient moving to another environment, the one or more physiological sensors 104 may be connected to an interface device 102 that can be conveniently brought with the patient to those other locations.

For example, the first environment may be a patient's hospital room within a hospital care unit, while the second environment is an imaging suite in a different part of the hospital. In other embodiments, the first environment can be a doctor's office while the second environment may be the patient's home. Although specific examples of different environments are provided, it should be appreciated that other possibilities and combinations are contemplated.

As shown in FIG. 3, the method 300 can further comprise, in a step 330, transmitting the second set of sensor data to a remote data storage unit 118 via the interface device 102. In embodiments, the transmission of the second set of sensor data may occur concurrently with its collection or in a piecemeal fashion (e.g., at designated times of the day, days of the week, etc.).

In embodiments, the method 300 can also comprise, in a step 340, storing the received second set of sensor data in a memory of the remote data storage unit 118. In embodiments, the remote data storage unit 118 is a server or collection of servers housed at an off-site location (i.e., away from the patient 106, the first environment, and/or the second environment). The memory of the remote data storage unit 118 can be variously embodied in one or more forms of machine-accessible and machine-readable memory, including, but not limited to, a non-transitory storage medium, a magnetic disk storage, an optical disk storage, an array of storage devices, a solid-state memory device, and the like, including combinations thereof.

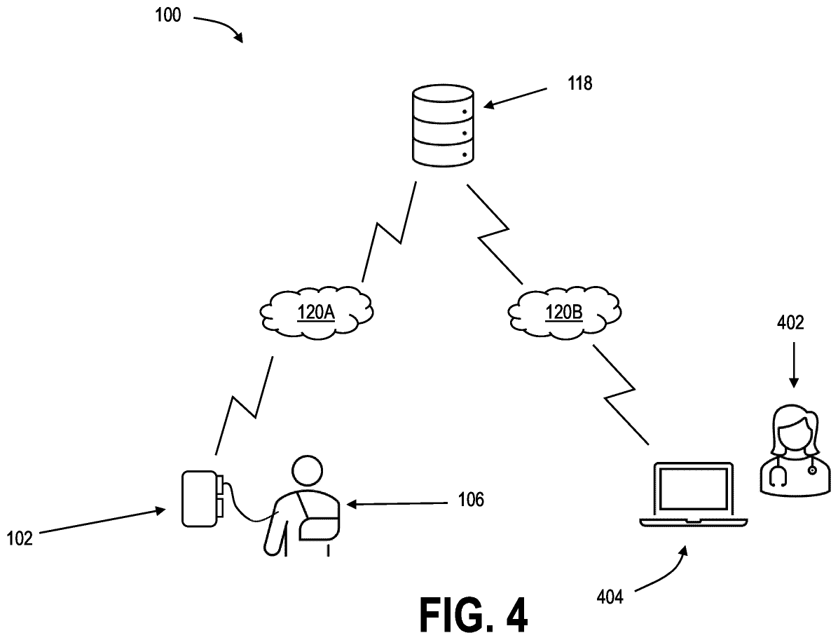

In further embodiments, the method 300 may include, in a step 350, receiving a request to access patient information for one or more patients (e.g., the patient 106) that is stored in the remote data storage unit 118. The request may be sent by a user of a client device and received by the remote data storage unit 118. For example, as shown in FIG. 4, a user 402 using a client device 404 may use an input device of the client device 404 to submit a request for patient information from the remote data storage unit 118. The request may be transmitted over a communications network 120B, which may be different or the same as communications network 120A used to transmit sensor data via the interface device 102.

In embodiments, the client device 404 can be variously embodied as, for example and without limitation, a mobile phone, desktop computer, laptop, portable digital assistants (PDAs), smart phones, tablets, netbooks, laptops, multi-processor systems, microprocessor-based or programmable consumer electronics, game consoles, set-top boxes, or other communication device that a user may use to access a network.

In specific embodiments, the client device 404 includes a networking component, a display, and/or a user input device. The networking component of the client device 404 may be similar to the network component 114, but further configured to receive patient information for a plurality of patients from the remote data storage unit 118. The display may be configured to display the patient information received from the remote data storage unit 118. And the user input device can include, but is not limited to, a keyboard, a touch screen configured to receive alphanumeric input, a photo-optical keyboard, or other alphanumeric input components, a mouse, a touchpad, a trackball, a joystick, a motion sensor, or other pointing instrument, a physical button, a touch screen that provides location and/or force of touches or touch gestures, or other tactile input components, a microphone, and/or the like.

In response to receiving a request for patient information for one or more patients, the method 300 can include, in a step 360, transmitting the requested patient information from the remote data storage unit 118. In embodiments, the method 300 can also include, in a step 370, receiving said patient information at the client device 404. As shown in FIG. 4, the patient information may be communicated over a network 120B, which may be a wireless network, a wired network, or a combination thereof.

In particular embodiments, the patient information includes at least the second set of sensor data collected while the patient 106 is within the second environment. In further embodiments, the patient information includes location-based information generated by a location-based tracking system 124. In still further embodiments, the sensor data may include time stamps associated with the data that indicates when the data was collected. In such embodiments, the method 300 may include adjusting the time stamp associated with sensor data of at least the second set of sensor data collected based on a sensor-specific delay. That is, each of the one or more physiological sensors 104 may have a sensor-specific delay that is accounted for either by the interface device 102, the remote data storage unit 118 and/or the client device 404. In embodiments, accounting for the sensor-specific delays in each of the one or more physiological sensors 104 includes adjusting the time stamp in the sensor data associated with each physiological sensor 104.

In a step 380, the method 300 can include using the client device 404 to visualize, analyze, and/or otherwise review the patient information remotely from the patient 106. For example, in embodiments, the step 380 can include visualizing the patient information on a display screen of the client device 404.

As described herein, the method 300 enables the continued monitoring of one or more patients 106 using an interface device 102 without requiring a patient 106 to remove expertly-placed sensors. Put another way, an improved transition of patients (e.g., patient 106) from a first, high-acuity environment (e.g., a hospital) to a second, typically low-acuity environment (e.g., a home) is provided.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A system for monitoring sensor data associated with a patient, the system comprising:
one or more physiological sensors attached to a patient;
one or more smart cables, each smart cable comprising a universal connector configured to enable communication from different types of the one or more physiological sensors; and
an interface device configured to receive sensor data from the one or more physiological sensors via the one or more smart cables, the interface device including one or more universal ports configured to receive a corresponding universal connector of the one or more smart cables,
wherein the one or more physiological sensors are connectable to a patient monitor in a high acuity environment and to the interface device in a low acuity environment via the one or more smart cables, enabling transition of the patient from the high acuity environment to the low acuity environment while the one or more physiological sensors remain attached to the patient.

2. The system of claim 1, wherein each of the one or more the smart cables comprises a controller configured to enable communication of sensor data from a plurality of different sensor types.

3. The system of claim 2, wherein the one or more physiological sensors include at least a first sensor having a first sensor type and a second sensor having a second sensor type, wherein the first sensor type is different from the second sensor type.

4. The system of claim 1, wherein each of the one or more physiological sensors is connected to the universal port of the interface device via a universal connector of one of the one or more smart cables.

5. The system of claim 1, wherein the one or more physiological sensors includes at least one of a pulse oximetry sensor, an ECG sensor, a heart rate sensor, a respiration rate sensor, a body temperature sensor, an actigraphy sensor, and a capnography sensor.

6. The system of claim 1, further comprising a remote data storage unit in wireless communication with the interface device, wherein the interface device further comprises a networking component configured to transmit the sensor data, and wherein the remote data storage unit is configured to receive the sensor data transmitted from the interface device and store the received sensor data in a memory.

7. The system of claim 6, further comprising a client device in communication with the remote data storage unit, the client device comprising:
a networking component configured to receive patient information for a plurality of patients from the remote data storage unit; and
a display configured to display the patient information received from the remote data storage unit;
wherein the patient information includes sensor data for the plurality of patients stored in the memory of the remote data storage unit.

8. The system of claim 6, wherein the interface device further comprises a global positioning system receiver configured to determine a location of the interface device, and wherein the networking component is further configured to transmit the location of the interface device to the remote data storage unit.

9. The system of claim 1, wherein the one or more physiological sensors are configured to be attached to the patient and collect: (i) a first set of sensor data for the patient (106) over a first period of time while connected to the patient monitor in proximity to the patient; and (ii) a second set of sensor data for the patient over a second period of time while connected to the interface device.

10. A method for monitoring a patient via one or more physiological sensors attached to the patient, while transitioning from a high acuity environment to a low acuity environment, the method comprising:
in the high acuity environment, collecting, via the one or more physiological sensors, a first set of sensor data for the patient over a first period of time, wherein the one or more physiological sensors are connected to a first patient monitor in proximity to the patient; and
in the low acuity environment, collecting, via the one or more physiological sensors, a second set of sensor data for the patient over a second period of time, wherein the one or more physiological sensors are connected to an interface device via one or more smart cables, wherein each smart cable comprises a universal connector configured to enable communication from different types of the one or more physiological sensors to the interface device.

11. The method of claim 10, further comprising:
transmitting, via the interface device, the second set of sensor data to a remote data storage unit; and
storing, in a memory of the remote data storage unit, at least the second set of sensor data, wherein the memory of the remote data storage unit comprises patient information for a plurality of patients.

12. The method of claim 11, further comprising:
receiving, at a client device, patient information from the remote data storage unit, wherein the patient information includes at least the second set of sensor data for the patient; and
visualizing, on a display of the client device, the patient information.

13. The method of claim 12, further comprising:
receiving, via a user input device of the client device, a request for the patient information of the patient, wherein the patient is selected from a plurality of patients for whom patient information is stored in the remote data storage unit; and
transmitting, from the remote data storage unit, the patient information for the patient in response to receiving the request.

14. The method of claim 12, further comprising:
adjusting a time stamp associated with sensor data of the second set of sensor data collected using at least one of the one or more physiological sensors, wherein the time stamp of the sensor data is adjusted based on a sensor-specific delay; and
visualizing, on a display of the client device, the patient information, wherein the patient information includes the sensor data of the second set of sensor data having the adjusted time stamp.

15. The method of claim 10, wherein the one or more physiological sensors includes at least a first sensor having a first sensor type and a second sensor having a second sensor type that is different from the first sensor type, and wherein the second set of sensor data comprises sensor data from at least the first and second sensors collected by the interface device via the one or more smart cables.
